(19) United States
(12) Patent Application Publication
(10) Pub. No.: US 2005/0165364 A1
(43) Pub. Date: Jul. 28, 2005

(54) Valved catheter to bypass connector

(76) Inventors: Kristian DiMatteo, Waltham, MA (US); Brett T. Haarala, Framingham, MA (US)

Correspondence Address: Patrick J. Fay, Esq.;FAY KAPLUN & MARCIN, LLP, Suite 702, 150 Broadway, New York, NY, 10038, US

(21) Appl. No.: 10/762,715

(22) Filed: Jan. 22, 2004

(51) Int. Cl. A61M 5/00

(52) U.S. Cl. 604/246; 604/118

(57) ABSTRACT

A connector for placement between an injector of contrast media and a venous catheter is described. The connector is adapted to protect the body of the catheter and the valve closing the catheter from excessive pressure and flow rate of the fluid being injected. The connector includes a valve bypass element to open the valve and pass the fluid beyond the valve, and a pressure control element to maintain the flow pressure below a selected value.

### BACKGROUND OF THE INVENTION

Many medical procedures require repeated and prolonged access to a patient's vascular system. For example, during dialysis treatment blood may be removed from the body for external filtering and purification, to make up for the inability of the patient's kidneys to carry out that function. In this process, venous blood is extracted, processed in a dialysis machine and returned to the patient. The dialysis machine purifies the blood by diffusing harmful compounds through membranes, and may add to the blood therapeutic agents, nutrients etc., as required before returning it to the patient's body. Typically the blood is extracted from a source vein (e.g., the vena cava) through a catheter sutured to the skin with a distal needle of the catheter penetrating the source vein.

Such semi-permanently implanted catheters are generally selected to be as small and thin as possible, to simplify the insertion procedure and to reduce discomfort to the patient. Accordingly, the structural strength of these catheters has been limited by their size and in particular by the thickness and materials forming the catheter's walls. The catheters' dimensions and structure in turn limit the flow rates and pressures of fluid that can pass therethrough without damage. If the maximum pressure of the catheter (the burst pressure) or the maximum flow rate is exceeded, the catheter may be damaged or may completely fail. This can be a serious problem, since a failure may result in the catheter's contents spilling within the body.

Valves have been used to seal proximal ends of such catheters when not in use. One common type of valve used is a Pressure Actuated Safety Valve (PASV), designed to open when a fluid pressure in the catheter exceeds a preselected threshold. PASV's may be damaged by high flow rates of the fluid impinging thereon. In addition, pressures substantially in excess of the pressure necessary to open the PASV may damage these valves rendering them incapable of fully closing when the pressure is withdrawn. This may allow bodily fluids to leak past the valve.

Furthermore, certain types of fluids administered require specialized procedures to avoid injuring the patient. For example, one such fluid is contrast media used to improve visualization of blood vessels and other biological structures within the patient's body during fluoroscopy, radiology, or other imaging processes. Contrast media is a liquid that is opaque to the visualization method used, so that body lumens containing the media will appear distinct from other tissues. Typically, contrast media is introduced into the body using a separate catheter designed to withstand high injection pressures, since the contrast media is best introduced at relatively high flow rates and pressures. A power injector as well as a conventional syringe may be used to inject contrast media through the catheter at an optimum flow rate.

### SUMMARY OF THE INVENTION

In one aspect, the present invention is directed to a connector for injecting fluid to a catheter including a valve therein, comprising an attachment portion adapted to fluidly couple to a source of pressurized fluid and a bypass element fluidly connected to the attachment portion, the bypass element being adapted to open a valve of the catheter in combination with an overpressure control element adapted to maintain a pressure of fluid within the connector below a predetermined threshold level.

### BRIEF DESCRIPTION OF THE DRAWINGS

FIG. 1 is a diagram showing an embodiment of the connector according to the present invention;

FIG. 2 is a diagram showing a detail of a bypass element according to an embodiment of the present invention, as inserted in a valve of a catheter;

FIG. 3 is a diagram of another embodiment of the connector according to the present invention, including an extension tube; and

FIG. 4 is a diagram showing a detail of an extension tube with a collection jacket, according to an embodiment of the present invention.

### DETAILED DESCRIPTION

The present invention may be further understood with reference to the following description and the appended drawings, wherein like elements are referred to with the same reference numerals. The invention is related to medical devices that are used to connect a source of pressurized fluid to a valved catheter, without damaging the valve or the catheter. More specifically, the invention relates to a fluid coupler used to protect a catheter from damage due to the introduction of fluid at a high flow rate and/or pressure.

Semi-permanently placed catheters may be useful for a variety of medical procedures which require repeated access to a patient's vascular system in addition to the dialysis treatments mentioned above. For example, chemotherapy infusions may be repeated several times a week for extended periods of time. However, those of skill in the art will understand that the exemplary embodiment of the present invention, in addition to the use described for long term catheters, may also be used for short term catheters and peripherally inserted central catheters (“PICCs”). For safety reasons, as well as to improve the comfort of the patient, injections of these therapeutic agents may be better carried out with an implantable, semi-permanent vascular access catheter. Many other conditions that require chronic venous supply of therapeutic agents, nutrients, blood products or other fluids to the patient may also benefit from implantable access catheters, to avoid repeated insertion of a needle into the patient's blood vessels. Thus, although the following description focuses on dialysis, those skilled in the art will understand that the invention may be used in conjunction with any of a wide variety of procedures which require long term implantation of catheters within the body. For the most part, these applications require a slow introduction of fluid into the catheter and thus into the patient's vascular system. For example, the delivery of chemotherapy agents, drugs and of blood products typically use low flow rates through the catheter. However, other procedures such as kidney dialysis may benefit from higher flow rates to minimize the amount of time the patient has to spend at a medical facility. However, in these cases the flow rates and accompanying pressures through the catheter must be maintained below levels which will damage the catheter and/or its components.

As described above, valves such as PASV's used to seal many of these catheters also have limitations on the amount of flow and on the maximum fluid pressure that can be withstood without causing damage. A common PASV comprises a flexible membrane with a slit extending therethrough. The slit is biased to a closed position by the flexibility of the membrane and/or additional biasing members when the catheter is not in use (i.e., when a fluid pressure within the catheter is below a threshold level). When the catheter is in use, edges of the slit are pushed apart by the pressure of the fluid impinging on the membrane, and the fluid passes through the PASV. However, if the fluid is supplied at an excessive pressure or flow rate, the membrane may be damaged so that, when the pressure is reduced below the threshold, the PASV may be unable to adequately close the slit resulting in leakage of bodily fluids from the catheter and/or the entrance of contaminants into the patient's bloodstream therethrough.

The flow rate within the catheter is related to the pressure generated therewithin and, thus, the maximum flow rate allowable through a catheter is related to the burst pressure of the catheter. More specifically, the pressure exerted by the fluid is a function of the flow rate through the catheter, the fluid's viscosity and a cross sectional flow area of the catheter, among other variables. Accordingly, limitations on the fluid pressure and/or flow rate are often specified for various types of catheters, to ensure that the catheter will not be damaged during use by being exposed to excessive pressures/flow rates. Damage to the catheter may also result in injury to the patient's tissues adjoining the catheter if high pressure fluid escapes from the damaged catheter into body lumens.

Manufacturers of several types and sizes of commonly used midline and central line venous catheters specify a maximum infusion pressure of about 25 psi if damage to the catheter and associated components is to be avoided. Larger catheters may be able to withstand higher pressures, for example in the range of about 100 psi to about 300 psi, depending on the catheter's construction and on the materials forming the catheter body. For example, a 3 F catheter may be able to support combinations of flow rate and pressure of about 0.65 ml/sec at 125 psi and 0.56 ml/sec at 125 psi without exceeding the burst pressure, depending on the catheter's length. For 5 F catheters, the flow rate and pressure combinations may be about 4.2 ml/sec at 200 psi and 3.02 ml/sec at 170 psi, also based on the catheter's length. Combinations of about 9.52 ml/sec at 350 psi and 8.78 ml/sec at 330 psi can be withstood by 7 F catheters of different lengths. As would be understood by those skilled in the art, the material of the catheter, for example silicone or polyurethane, may also affect the burst strength of the catheter.

Conventionally, the injection of contrast media into the blood stream is carried out using catheters inserted for that specific purpose. However, if certain precautions are taken, central venous access catheters that have been implanted to carry out other medical procedures may also be used for the injection of contrast media. Embodiments of the present invention may be used to safely introduce into a valved catheter fluids at high pressure and high flow rate, while minimizing the possibility of damage to the catheter and valve and resulting injury to the patient.

By using a connector according to embodiments of the present invention between the implanted venous catheter and the contrast media injection device, it is possible to safely conduct imaging techniques without placing an additional specialized catheter in the patient. For example, injection pressures of between about 40 psi and 80 psi may be used with current valved venous catheters, without damage to the catheter while improved catheters may be able to withstand injection of contrast media at pressures from about 100 psi to about 300 psi. As will be described below, the connector according to the present invention shields the catheter's valve from damage, and prevents pressure spikes from rupturing the catheter.

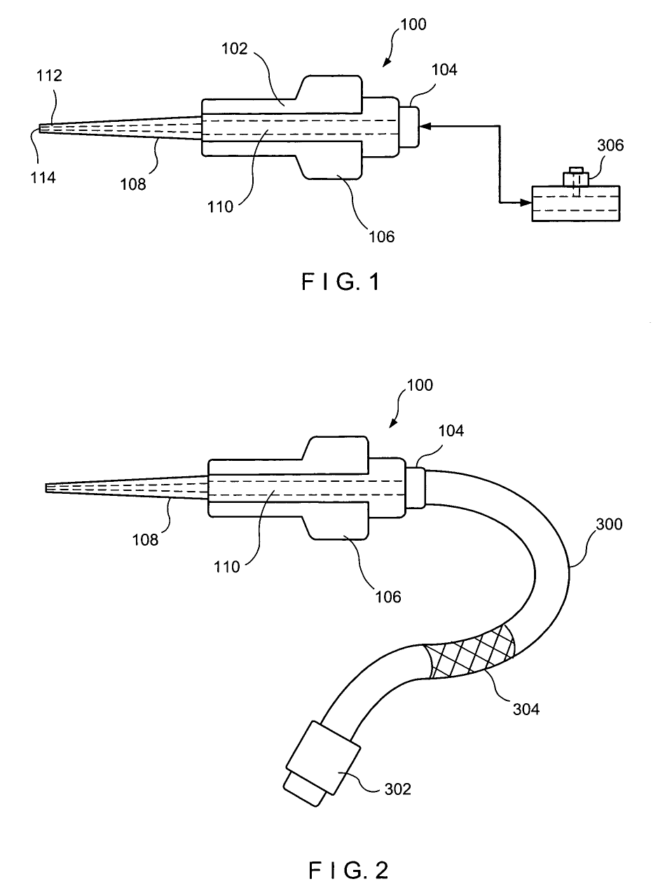

As shown in FIG. 1, a connector 100 according to the invention is provided, which enables injection of a contrast media at elevated pressure into a patient's vascular system through a valved venous catheter. In the exemplary embodiment shown, the connector comprises a connector body 102 which may be, for example, a male luer connector alone or together with a female luer connector. The connector body 102 may include extensions 106 designed to facilitate grasping and twisting the device in order to attach and detach the connector body 102 from other devices having matching attachment elements. The exemplary embodiment shown includes an attachment portion 104 at a proximal end of the connector body 102, which is designed to fluidly couple the connector 100 to a source of pressurized fluid to be injected into the patient. In one embodiment, the source may be a power injector used to inject contrast media during imaging procedures. Alternatively, different sources of pressurized fluid may include hand operated pumps, syringes, pressure vessels etc. Attachment portion 104 may be designed to interface with a conventional luer connection, as is known in the art.

At a distal end of the device, opposite from attachment portion 104, a bypass element is provided which ensures that the flow control valve of the catheter used for the injection is not damaged by the high pressure injection. The bypass element comprises an elongated tube-like portion that is inserted in the proximal end of the catheter and extends beyond the valve. For example, the tube like portion may have a length sufficient so that a distal tip thereof extends past the flexible slitted membrane of a PASV valve located in a proximal portion of the catheter. As shown in FIG. 1, the exemplary bypass element may be a hypotube 108 having a distal tip 112. The hypotube 108 may be similar to a syringe needle, with inner and outer diameters selected to provide a desired flow rate through the tube. The outer diameter is also selected to fit into an opening in the PASV when the PASV is in the open position. An orifice 114 is be disposed at the distal end of the hypotube 108, and a fluid connection 110 may be provided along the length of the connector 100 to allow passage of fluid between the orifice 114 and the attachment portion 104. It will be apparent to those skilled in the art that different configurations of the attachment portion, body and bypass element may be used without affecting the functionality of the device. For example, the elements may be mounted at an angle to each other instead of along a common longitudinal axis.

A more detailed understanding of the functioning of the bypass element according to the present invention can be obtained by referring to the diagram shown in FIG. 2. The bypass element comprising hypotube 108 is shown inserted into the proximal end 212 of a catheter 200. A valve 202, for example a PASV, is located within proximal end 212, to prevent fluids from entering or escaping the catheter 200 as described above. The valve 202 comprises inlets 204, 206 and a slitted flow control membrane 208 which allows fluid to flow through the valve 200 only under predetermined conditions. As would be understood by those skilled in the art, the flow control membrane 208 may be a flexible polymeric membrane with one or more slits formed therethrough. As described above, when a fluid of at least a threshold pressure impinges on the membrane 208, edges of the slit separate from one another to form an opening 210 and, when the fluid pressure drops below the threshold level, the slit is closed to prevent fluid flow therethrough.

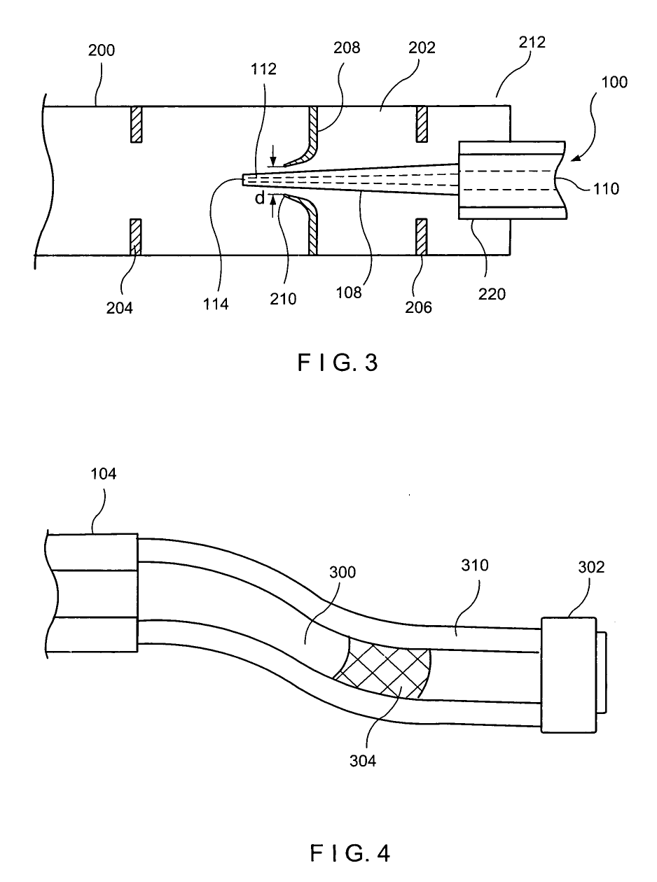

As shown in FIG. 3, the hypotube 108 is sized so that it extends through the slit, past the flow control membrane 208 when the connector 100 is in its operative position attached to the proximal end 212 of the catheter 200. The diameter of the hypotube 108 is preferably selected to be smaller than a maximum opening size “d” of membrane 208, so insertion of the hypotube 108 therethrough does not damage the membrane 208. As shown, the tip 112 of the hypotube 108 extends beyond opening 210 so that the fluid exiting the orifice 114 is injected beyond the membrane 208, and cannot damage it. The tip 112 may be shaped (e.g., rounded) to facilitate the opening of the slit without damaging the membrane 208. In some embodiments, the hypotube 108 may extend beyond the outlet 204 of the valve 200, to further prevent damage by reducing the effects of any back flow on the flow control membrane 208. The distal end 220 of the connector 100 may be shaped to form a seal with the proximal end 212 of the catheter 200, when the connector 100 is placed therein in the operative position to minimize leakage from the open membrane 208 and maintain a sterile environment in and around the catheter 200.

Providing a bypass element such as the hypotube 108 enables the connector 100 to avoid damaging a flow control valve located near the proximal end of a medical venous catheter. However, a second problem can manifest itself when high pressure fluid is injected through such catheters. As described above, medical catheters tend to be small and flexible, and thus have a relatively low resistance to internal pressures exerted by fluids flowing therethrough. When a fluid, for example contrast media, is injected into a catheter using a power injector or a syringe, the resulting fluid pressure may exceed the burst pressure of the catheter, and the catheter may be damaged or destroyed. If the fluid injection is done manually, as when using a syringe, it is difficult to precisely gauge the pressure applied, because it is affected by many variables. To preclude inadvertent damage, the present invention provides devices that prevent the creation of an overpressure condition in the catheter during fluid injection. These devices may work, for example, by providing an alternative path for the high pressure fluids, or by releasing pressurized fluid from the connector.

In one exemplary embodiment, an overpressure control device incorporated into the connector 100 may comprise a pressure relief valve that is set to open at a preselected pressure threshold. The threshold may represent a pressure level lower than the burst pressure of the catheter connected thereto, so that the pressure relief valve will open and reduce the injection pressure before any damage to the catheter takes place. For example, a spring loaded pressure relief valve 306 may be located near the attachment portion 104, downstream of the source of pressurized fluid. It will be apparent to those of skill in the art that a pressure relief valve analogous to the valve 306 may be placed at other locations on or near the connector 100, so long as it is maintained in fluid communication with the fluid conduit 110.

In a different embodiment according to the present invention, the overpressure control element of the connector according to the invention may be an extension tube connected to the attachment portion 104. In an exemplary embodiment shown in FIG. 3, a flexible extension tube 300 is used to connect the attachment portion 104 of the connector 100 to a source of fluid with an optional second attachment portion 302 provided at the proximal end of the extension tube 300 to interface with a power injector, syringe or other manual or mechanical injection system.

The extension tube 300 comprises a controlled failure element 304 which is designed to burst when subject to an internal pressure of at least a threshold level wherein the threshold level is selected to be lower than the burst pressure of the catheter to be attached to the connector 100. In this manner, if an excessive fluid pressure is provided by the fluid source connected to the second attachment portion 302, the failure element 304 will burst before the fluid can reach the catheter so that the pressure within the catheter and the connector is maintained at a level that is safe for the catheter. In a different embodiment, a failure element analogous to the failure element 304 may be incorporated at another location on or near the connector 100, in fluid communication with the conduit 110. For example, a reduced strength section may be incorporated within a separate dead ending tube extending from the connector 100, or as a portion of the connector body 102 as would be understood by those skilled in the art.

When the failure element incorporated in the connector 100 bursts, the fluid contained therein is allowed to escape and spill into the surrounding area. Accordingly, the failure element may be positioned at a location where it is not likely to cause a spill of the fluid on the patient or within a body lumen containing the catheter. For example, by incorporating the failure element in an extension tube as shown in FIG. 3, the spilled fluid can be kept away from the patient. In addition, a fluid capture structure may be used to contain any spilled fluid within a designated area. As shown in FIG. 4, an external jacket 310 may be placed around the extension tube 300 to shroud it from the surrounding environment. Thus, if the failure element 304 breaks under excessive pressure, the jacket 310 contains the spilled fluid in a small area preventing problems associated with its contact with any of the other substances or items in the area.

A jacket similar to that shown in FIG. 4, or another type of shroud may also be employed in designs where the failure element is located elsewhere on the connector 100. For example, a containment jacket may also be placed around a pressure relief valve that may be used as part of the overpressure control element of the connector so that, when the pressure relief valve releases fluid to maintain a pre-selected pressure in the connector and in the catheter, the fluid is captured within the shroud, and is prevented from contaminating the patient and the operating area.

The present invention has been described with reference to specific embodiments, and more specifically to a connector used for injecting contrast media into a valved venous catheter. However, other embodiments may be devised that are applicable to other medical devices, without departing from the scope of the invention. For example, any of the connectors described herein may also be employed with catheters that do not include valves. Accordingly, various modifications and changes may be made to the embodiments, without departing from the broadest spirit and scope of the present invention as set forth in the claims that follow. The specification and drawings are accordingly to be regarded in an illustrative rather than restrictive sense.

1. A connector for injecting fluid to a catheter, comprising:
an attachment portion adapted to fluidly couple to a source of pressurized fluid;
a bypass element fluidly connected to the attachment portion, the bypass element being adapted to open a valve of the catheter; and
an overpressure control element adapted to maintain a pressure of fluid within the connector below a predetermined threshold level.

2. The connector according to claim 1, wherein the bypass element comprises an elongated tubular component insertable into the catheter through a valve of the catheter.

3. The connector according to claim 2, wherein the elongated tubular component has a diameter selected to fit in a flow opening of the valve of the catheter.

4. The connector according to claim 2, wherein the elongated tubular component is hypotube.

5. The connector according to claim 2, wherein the elongated tubular component includes an outlet which, when the elongated tubular component is inserted into the catheter through the valve, is located distally of the valve.

6. The connector according to claim 2, wherein the bypass element directs fluid into the catheter so that the fluid passes through the catheter without passing through the valve.

7. The connector according to claim 1, wherein the overpressure control element comprises a pressure relief valve.

8. The connector according to claim 1, wherein the overpressure control element comprises a controlled failure element designed to fail when a fluid pressure therein reaches the threshold level.

9. The connector according to claim 8, wherein the controlled failure element is an extension tube.

10. The connector according to claim 1, further comprising an external collection jacked disposed around the overpressure control element.

11. The connector according to claim 1, wherein the bypass element is adapted to open a pressure actuated safety valve of a venous catheter.

12. The connector according to claim 1, wherein the attachment portion is adapted to connect to a contrast media power injection system.

13. The connector according to claim 1, wherein the threshold level is selected to be less than a burst pressure of a catheter with which the connector is to be used.

14. The connector according to claim 13, wherein the threshold level is approximately 300 psi.

15. The connector according to claim 14, wherein the threshold level is approximately 100 psi.

16. The connector according to claim 13, wherein the threshold level is approximately 80 psi.

17. The connector according to claim 16, wherein the threshold level is approximately 40 psi.

18. A fluid coupler comprising:
an elongated tube extending between a first end adapted for fluid connection to a power injector and a second end adapted for fluid connection to a catheter, the second end being insertable into the catheter beyond a proximal part thereof so that fluid from passes through the fluid coupler into the catheter to a distal end thereof without passing through the proximal part; and
a pressure control element adapted to limit a fluid pressure within the coupler to a predetermined threshold level.

19. The coupler according to claim 18, wherein the elongated tube is a hypotube.

20. The coupler according to claim 18, wherein the proximal part of the catheter includes a valve and wherein the elongated tube is adapted to open the valve when inserted in the catheter.

21. The coupler according to claim 18, wherein the pressure control element comprises a section having a burst pressure lower than a burst pressure of the catheter.

22. The coupler according to claim 18, wherein the pressure control element comprises an extension tube connected to the first end.

23. The coupler according to claim 18, wherein the pressure control element comprises a pressure relief valve.

24. The coupler according to claim 18, further comprising a fluid collection jacket surrounding the pressure control element.
